(19) United States
(12) Patent Application Publication
(10) Pub. No.: US 2017/0333472 A1
(43) Pub. Date: Nov. 23, 2017

(54) BACTERICIDAL COMPOSITION

(71) Applicant: 3M INNOVATIVE PROPERTIES COMPANY, St. Paul, MN (US)

(72) Inventors: Masashi Okuno, Kanagawa (JP); Matthew T. Scholz, Woodbury, MN (US)

(21) Appl. No.: 15/529,642

(22) PCT Filed: Dec. 22, 2015
(86) PCT No.: PCT/US2015/067395

§ 371 Date: May 25, 2017

Related U.S. Application Data
(60) Provisional application No. 62/098,029, filed on Dec. 30, 2014.

(51) Int. Cl. A61K 31/79; A61K 47/14; A61K 33/18; A61K 47/38; A61K 9/00

(52) U.S. Cl. CPC A61K 31/79; A61K 47/38; A61K 9/0014; A61K 33/18; A61K 47/14

(57) ABSTRACT

A bactericidal composition comprising a carboxy group-containing cellulose ester or carboxy group-containing cellulose ether, a bactericide, alcohol, and water. Some embodiments further comprise a plasticizer. The compositions described herein may be used to disinfect the surface of skin in preparation for surgical procedures.

### CROSS REFERENCE TO RELATED APPLICATIONS

This application claims priority to U.S. Provisional Patent Application No. 62/098,029, filed Dec. 30, 2014, the disclosure of which is incorporated by reference in its entirety herein.

### FIELD OF THE INVENTION

The present invention relates to a bactericidal composition.

### BACKGROUND

In order to reduce the risk of infection in patients, disinfecting the skin prior to performing invasive treatment such as surgery, anesthetic, or catheter insertion has been a standardized medical practice among clinicians for some time.

The practice of disinfecting the skin prior to invasive treatment such as a surgical operation, catheter treatment, or needle puncture in order to reduce the possibility of infection is called “prepping.” A composition used as an antiseptic at the time of prepping is typically called a “prepping product.” A known example of a prepping product is a bactericidal alcohol gel preparation containing prescribed amounts of alcohol, iodine, and a thickener.

Surgical incise drapes are often used in addition to a prepping product prior to performing invasive treatment. Surgical incise drapes are typically comprised of a film substrate coated with an antimicrobial composition. The primary function of a surgical drape is to prevent the infection of the surgical site by preventing microbial flora on the skin surface from migrating to the surgical site.

### SUMMARY OF THE INVENTION

As used herein, “Removal resistance” means that the bactericidal composition of the present invention maintains a film shape adhered to the skin without being peeled or dissolved under the flow of water.

“Drape adhesive strength” means that the bactericidal composition of the present invention has sufficient adhesive strength to adhere a surgical drape and the skin coated with the composition under conditions in which an excessive amount of moisture is present.

“Bactericidal action” means that the number of residual bacteria colonies is small when the number of colonies is measured with a cup scrub method after the composition is applied to the skin.

### DETAILED DESCRIPTION

The present invention provides a bactericidal composition containing a carboxy group-containing cellulose ester or carboxy group-containing cellulose ether, a bactericide, alcohol, and water. In some embodiments, the composition further contains a plasticizer.

During surgical and other medical procedures, the area to which a prepping product is applied is often exposed to an enormous amount of moisture from water or saline used by a clinician during a given procedure, or blood or the like flowing from the surgical site (this state will also be called a “liquid loading environment” hereafter). Thus, to be effective, it is important that a prepping product maintain good adhesion to a patients' skin in a liquid loading environment.

The bactericide composition of the present invention can be used as a prepping product and demonstrates a high bactericidal effect, adheres to the skin very well, and the bactericide contained in the composition is unlikely to be removed even in a liquid loading environment (such a characteristic will also be called “removal resistance” hereafter). Moreover, the bactericidal composition described herein has a film-forming ability, so a film with excellent bactericidal properties and excellent removal resistance is formed when applied to the skin, and an adhesive film (this may be a surgical drape containing a bactericide or a surgical drape not containing a bactericide) may be further laminated over this film. Even when used with a surgical drape, the bactericidal composition described herein demonstrates excellent bactericidal effect, adhesion to skin, and removal resistance.

In a liquid loading environment, the adhesive strength with respect to a surgical drape (also called the “drape adhesive strength” hereafter) typically decreases, which may ultimately cause the removal of the surgical drape from the skin. The removal of the surgical drape from the skin produces a space into which microorganisms can migrate between the drape and the skin, which may dramatically diminish the infection-preventing effect of the surgical drape.

The bactericidal composition disclosed herein functions as a preoperative skin disinfectant and demonstrates removal resistance in a liquid loading environment such that it may be effectively used with a surgical incise drape. In addition, when the bactericidal composition further contains a plasticizer the composition also demonstrates improved drape adhesive strength.

With the bactericidal composition, it is possible to provide a persistent film for forming a preoperative skin disinfectant having a sufficient bactericidal action, removal resistance in a liquid loading environment, and excellent drape adhesive strength. That is, when applied to a surgical site, the bactericidal composition forms an extremely persistent film having a bactericidal action on the skin surface, and the bactericide is unlikely to be removed in an environment exposed to water or saline used in surgical operations. In an aspect in which the bactericidal composition of the described further contains a plasticizer, it is also possible to suppress the removal of a surgical drape from the skin.

In addition, with the bactericidal composition disclosed herein, it is possible to form a persistent film having a bactericidal action with only this bactericidal composition. Furthermore, in an aspect in which the bactericidal composition further contains a plasticizer, it is also possible to increase the adhesive strength between the skin and a surgical drape, such that the composition exhibits a synergistic effect when used in combination with a surgical incise drape. Accordingly, the surgical drape used in combination with the bactericidal composition may be a surgical drape that does not have a bactericidal action or a surgical drape having a bactericidal action (i.e., a drape containing an antimicrobial). When the bactericidal composition of the preset invention and a surgical drape having a bactericidal action are used in combination, the overall bactericidal action improves.

### Carboxy Group-Containing Ester

Examples of carboxy group-containing cellulose ester polymers suitable for use in the compositions disclosed herein include monoesters of a cellulose derivative such as hydroxyalkyl alkyl cellulose and a poly carboxylic acid. The carboxy group-containing cellulose ester is preferably a dicarboxylic acid monoester of hydroxyalkyl alkyl cellulose. Examples of dicarboxylic acid monoesters of hydroxyalkyl alkyl cellulose include hydroxyalkyl alkyl cellulose phthalates such as hydroxypropyl methyl cellulose phthalate and hydroxyalkyl alkyl cellulose acetate succinates such as hydroxypropyl cellulose acetate succinate. In addition, the carboxy group-containing cellulose ester preferably further contains an aromatic ring in its chemical formula in order to further improve the persistence of the bactericide. Examples of such carboxy group-containing cellulose esters include aromatic dicarboxylic acid monoesters of hydroxyalkyl alkyl cellulose, and of these, hydroxyalkyl alkyl cellulose phthalates such as hydroxypropyl methyl cellulose phthalate are particularly preferable (HPMCP). HPMCP (available from Shin-Etsu Chemical Co., Ltd.) or the like can be used for hydroxypropyl methyl cellulose phthalate, and Shin-Etsu AQOAT (available from Shin-Etsu Chemical Co., Ltd.) can be used for hydroxypropyl cellulose acetate succinate.

In an embodiment, the upper limit of the carboxy group-containing cellulose ester polymer content is at most 10%, in a further embodiment, the upper limit is at most 8%, and in a further embodiment the upper limit is at most 5% based on the weight of the entire bactericidal composition.

In an embodiment, the lower limit of the carboxy group-containing cellulose ester content is at least 0.1%, in a further embodiment the lower limit is at least 0.5%, and in a further embodiment the lower limit is at least 1% based on the weight of the entire bactericidal composition.

### Carboxy Group-Containing Cellulose Ether

The carboxy group-containing cellulose ether is not particularly limited as long as it is a cellulose ether containing a carboxy group in its chemical formula. Examples of carboxy group-containing cellulose ethers include compounds obtained by esterifying a carboxylic acid having a halogen atom to a cellulose derivative such as hydroxyalkyl alkyl cellulose.

Specific examples include carboxymethyl cellulose, sold as Daicel CMC (available from Daicel FineChem Ltd.) and Cellogen (available from Dai-ichi Kogyo Seiyaku Co., Ltd.).

In an embodiment, the upper limit of the carboxy group-containing cellulose ether content is at most 10%, in another embodiment, at most 8%, and in a further embodiment, at most 5% based on the weight of the entire bactericidal composition. In an embodiment, the lower limit of the carboxy group-containing cellulose ether content is preferably at least 0.1 mass %, more preferably at least 0.5 mass %, and even more preferably at least 1 mass % based on the mass of the entire bactericidal composition from the perspective of being able to provide a bactericidal composition with excellent removal resistance.

In some embodiments of the bactericidal composition, a carboxy group-containing cellulose ester and a carboxy group-containing cellulose ether may be used in combination.

### Bactericide

The bactericide contained in the bactericidal composition may be any additive having a bactericidal action. Examples of bactericides include iodine, triiodide salts, iodophors such as iodine complexed to polyethylene glycol (PEG), PEG derivatives such as PEG surfactants, polymers and copolymers dervived from N-vinylpyrrolidone such as povidone-iodine and particularly povidone iodine USP, sodium hypochlorite, phenols, cresols, triclosan, parachlorometaxylenol, isopropyl methyl phenol and other phenolic antiseptics disclosed in U.S. Patent Application Publication No. 2006/0052452; quaternary ammonium bacteriacidal surfactants such as benzalkonium chloride, benzethonium chloride, cetylpyridinium chloride, and octenidine dihydrochloride, as well as biguanides such as chlorhexidine salts including chlorhexidine gluconate, alexidine, polymeric biguanides such as polyhexamethylene biguanide other cationic antiseptics disclosed in U.S. Patent Application Publication No. 2006/0051385, antimicrobial lipids such as C8-C12 fatty acid esters of glycerin and propylene glycol, C7-C12 alkyl ethers of glycerin, C6-c12 1,2 alkane diols and other antimicrobial lipids disclosed in U.S. Patent Application Publication No. 2005/0089539, alkyl diaminoethyl glycine hydrochloride, acrinol hydrate, and natural oils and peroxides including hydrogen peroxide as well as those disclosed in U.S. Patent Application Publication No. 2006/0051384 and combinations thereof The above referenced patent applications are incorporated by reference herein in their entirety.

In an embodiment, the bactericide is povidone-iodine, which has a wide antibacterial spectrum. Povidone-iodine is desirable from the perspective that it is easy to confirm the presence or absence of the bactericide since the site to which it is applied is colored. For bactericide compositions that are colorless or do not have enough color to be seen on most skin tones, a compatible dye may be used.

The bactericide content in one embodiment is from about 0.05% to about 20% based on the weight of the entire bactericide composition, in a further embodiment, from about 0.25% to about 15%, and in another embodiment, from about 0.5% to about 10%. Preferably the bactericide is present at greater than about 1% and more preferably at least about 2%. In an embodiment, povidone iodine is used at about 5-10% by weight of the composition.

### Alcohol

The alcohol contained in the bactericidal composition is not particularly limited as long as the carboxy group-containing cellulose ester or carboxy group-containing cellulose ether and the bactericide can be sufficiently mixed with water. Examples of the alcohol include ethanol, propanol, and isopropanol. The bactericidal composition of this embodiment is used by drying after being applied so as to form a film, so an alcohol with a low boiling point and low skin irritation is preferable. In an embodiment, ethanol is used as the alcohol.

In one embodiment, the alcohol content is from about 10% to about 90%, in a further embodiment, the alcohol content is about 20% to about 80% based on the weight of the entire bactericidal composition. In one embodiment the alcohol content is greater than about 40% by weight, and in a further embodiment greater than about 50% by weight and in another embodiment greater than 60% by weight.

In some embodiments, the alcohol content is about 1.5 to 4 times the weight of the water.

### Water

Various sources of water may be used in the composition of the present invention, so long as the water is properly sterilized. In one embodiment, the water content is about 5% to about 50% based on the mass of the entire bactericidal composition.

### Plasticizer

The bactericidal composition may further contain a plasticizer. The plasticizer decreases the glass transition temperature of the carboxy group-containing cellulose ester or carboxy group-containing cellulose ether as measured by differential scanning calorimetry of the polymer and plasticizer alone. The addition of a plasticizer additionally provides plasticity, i.e., decreases the modulus of the film. In some embodiments, hydrophobic oils are used as a plasticizer. Examples of hydrophobic plasticizers include diisopropyl adipate, diisobutyl adipate, diisopropyl sebacate, isopropyl myristate, and tri(capryl/capric acid) glycerin. In some embodiments, a dicarboxylic acid ester such as C3-C8 alkyl alcohol esters of C4-C12 alkyl dicarboxylic acids such as diisopropyl adipate, diisobutyl adipate, or diisopropyl sebacate is used as a plasticizer.

Plasticizers may further be selected from short chain alkyl or aryl esters (C1-C6) of long chain straight or branched chain alkyl or alkenyl alcohols or acids (C8-C36) and their polyethoxylated derivatives; short chain alkyl or aryl esters (C1-C6) of C4-C12 diacids or diols optionally substituted in available positions by —OH; C4-C18 alkyl or C6-C24 aryl esters of polyhydroxy compounds such as glycerol, polyglycerin having 2-10 glycerin units, sugar alcohols such as xylitol and sorbitol, pentaerythritol, ethylene glycol, propylene glycol, as well as polyethoxylated derivatives of these and polyethylene glycol; C12-C22 alkyl esters or ethers of polypropylene glycol; C1-C8 alkyl esters of polyacids such as citric acid, trimellitic acid, phthalic acids, succinic acid, malic acid, tartaric acid, and C4-C12 diacids; C12-C22 alkyl esters or ethers of polypropylene glycol/polyethylene glycol copolymer; long chain (C8-C36) alkyl and alkenyl esters of long straight or branched chain alkyl or alkenyl alcohols or acids; long chain (C8-C36) alkyl and alkenyl amides of long straight or branched chain (C8-C36) alkyl or alkenyl amines or acids.

In one embodiment, the plasticizer is present from about 1% to about 30% based on the weight of the bacterial composition. In a further embodiment, the plasticizer is present from about 5% to about 20%. As demonstrated in the examples below, the addition of a plasticizer improves persistence and drape adhesive strength.

The bactericidal composition can be produced by mixing a carboxy group-containing cellulose ester or carboxy group-containing cellulose ether, a bactericide, alcohol, and water. In addition, when the bactericide is povidone-iodine, it is preferable to add povidone-iodine after mixing the carboxy group-containing cellulose ester or carboxy group-containing cellulose ether, alcohol, and water. The temperature, mixing method, and the like when producing the bactericidal composition described above may be changed appropriately using methods known to persons having ordinary skill in the art.

### EXAMPLES

The present invention will be described in further detail hereinafter using working examples and comparative examples, but the present invention is not limited to these examples.

After the ingredients other than povidone-iodine were mixed in accordance with the content of Table 1, povidone-iodine was added to obtain the compositions of Working Examples 1 to 3 and Comparative Examples 1 to 4. Unless specified otherwise, the numerical values in Table 1 refer to “content (mass %)”.

TABLE 1

| | Working | Working | Working | Comp. | Comp. | Comp. | Comp. |
|---|---|---|---|---|---|---|---|
| Composition | Ex. 1 | Ex. 2 | Ex. 3 | Ex. 1 | Ex. 2 | Ex. 3 | Ex. 4 |
| Povidone-iodine | 10 | 10 | 10 | 10 | 10 | 10 | 10 |
| Hydroxypropyl methyl | 1 | 1 | 5 | — | — | — | — |
| cellulose phthalate | | | | | | | |
| Hydroxypropyl | — | — | — | 1 | 1 | — | — |
| cellulose | | | | | | | |
| Hydroxyethyl | — | — | — | — | — | 1 | 1 |
| cellulose | | | | | | | |
| Diisopropyl | — | 12 | 12 | — | — | — | — |
| adipate | | | | | | | |
| Propylene glycol | — | — | — | — | 12 | — | 12 |
| Ethanol | 50 | 50 | 50 | 50 | 50 | — | — |
| Water | 39 | 27 | 23 | 39 | 27 | 39 | 27 |
| Total | 100 | 100 | 100 | 100 | 100 | 100 | 100 |

### Persistence Test 1

The persistence of the bactericidal compositions was evaluated with the following method. The compositions of Working Examples 1 and 2, the compositions of Comparative Examples 1 and 3, and ISODINE (registered trademark) (10% povidone-iodine aqueous solution, available from Meiji Seika Pharma Co., Ltd.; Reference Example 1) were respectively applied uniformly to a 2 cm×2 cm area of the dorsal skin of a euthanized pig in quantities of 200 μL. After the applied composition was completely dried, the application site was held at an angle of approximately 45° from the vertical direction. Next, tap water was run from a height of approximately 15 cm from the application site at 23 to 24° C. and at a flow rate of approximately 2.5 L/min so that the water touched the portion directly above the composition and flowed over the application site. The time until the brown color of iodine completely disappeared visually was measured and recorded as the persistence in Table 2.

TABLE 2

| | Persistence |
|---|---|
| | (seconds) |
| Working Example 1 | 27 |
| Working Example 2 | 55 |
| Comparative Example 1 | 19 |
| Comparative Example 3 | 18 |
| Reference example 1 | 9 |

The compositions of Working Examples 1 and 2 containing carboxy group-containing cellulose esters demonstrated persistence superior to that of the compositions of Comparative Examples 1 and 3 containing cellulose esters not containing carboxy groups. In addition, the time that the bactericide was retained was extended by approximately double in the composition of Working Example 2, which further contained a plasticizer, in comparison to the composition of Working Example 1 not containing a plasticizer. The compositions of the working examples and the comparative examples demonstrated bactericidal action persistence superior to that of the composition of Reference Example 1.

### Persistence Test 2

After the ingredients other than povidone-iodine were mixed in accordance with the content of Table 3, povidone-iodine was added to obtain the compositions of Working Examples 4 and 5 and the composition of Comparative Example 5. Unless specified otherwise, the numerical values in Table 3 refer to weight percent of the overall composition.

TABLE 3

| | Working | Working | Comparative |
|---|---|---|---|
| Composition | Example 4 | Example 5 | Example 5 |
| Povidone-iodine | 10 | 10 | 10 |
| Hydroxypropyl methyl | 1 | — | — |
| cellulose phthalate | | | |
| Hydroxypropyl methyl | — | 1 | — |
| cellulose acetate succinate | | | |
| Ethanol | 50 | 50 | 50 |
| Water | 39 | 39 | 40 |
| Total | 100% | 100% | 100% |

The persistence of the bactericidal compositions was evaluated with the following method. The compositions of Working Examples 4 and 5, the composition of Comparative

Example 5, and the composition of Reference Example 1 were respectively dropped onto the dorsal skin of a euthanized pig in quantities of 200 μL and uniformly spread over a 2 cm×2 cm area with a spatula. After the applied composition was completely dried (after at least 5 minutes), the application site was held at an angle of approximately 45° from the vertical direction. Next, tap water was run from a height of approximately 15 cm from the application site at 23 to 24° C. and at a flow rate of approximately 2.5 L/min so that the water touched the portion directly above the composition and flowed over the application site. The time until the brown color of iodine completely disappeared visually was measured and recorded as the persistence in Table 4.

TABLE 4

| | Persistence (seconds) |
|---|---|
| Working Example 4 | 21.0 |
| Working Example 5 | 11.3 |
| Comparative Example 5 | 8.7 |

The compositions of Working Examples 4 and 5 containing carboxy group-containing cellulose esters demonstrated persistence superior to that of the composition of Comparative Example 5 containing a cellulose ester not containing a carboxy group. In particular, the composition of Working Example 4 demonstrated persistence superior to that of the composition of Working Example 5.

### Surgical Drape Adhesion Test 1 in a Liquid Loading Environment

The composition of Working Example 2, the compositions of Comparative Examples 2 and 4, and the composition of Reference Example 1 were respectively applied uniformly to an approximately 4 cm×12 cm area of the dorsal skin of a euthanized pig and left to completely dry for 20 minutes. An IOBAN (registered trademark) 2 Antimicrobial Incise Drape (available from the 3M Company) was cut into a strip shape with a width of 0.5 inches (1.27 cm), and after this was adhered to the dried composition, the composition was crimped with a 4.5 pound (2.1 kg), 2 inch (5.1 cm) roller in order to realize a uniform adhesive pressure. Each strip was left to stand for 30 minutes in order to generate an appropriate amount of adhesion. Gauze immersed in saline was then adhered to the upper part of the strip, and this was left to stand for 20 minutes to produce a liquid loading environment. Next, each strip was peeled at a peeling angle of 90° and a speed of 12 inches/min (30.5 cm/min) using a Zwick Roell 7005 tensile strength measurement device. The average value of the strength (N) required to peel each sample over a length of at least 3 inches (7.6 cm) was recorded. At least four strips were evaluated for each composition, and the average values of the measured strengths are shown in Table 5.

TABLE 5

| | Drape adhesive strength |
|---|---|
| | (Newtons) |
| Working Example 2 | 0.3433 |
| Comparative Example 2 | 0.1780 |
| Comparative Example 4 | 0.1323 |
| Reference example 1 | 0.1595 |

As shown in Table 5, the drape adhesive strength of Working Example 2 was superior to the drape adhesive strength of Comparative Examples 2 and 4 and Reference Example 1.

### Surgical Drape Adhesion Test 2 in a Liquid Loading Environment

After the ingredients other than povidone-iodine were mixed in accordance with the content of Table 6, povidone-iodine was added to obtain the compositions of Working Examples 6 to 9. 3M DURAPREP Surgical Solution (registered trademark, available from the 3M Company) was used as Reference Example 2. Unless specified otherwise, the numerical values in Table 6 refer to weight percent.

TABLE 6

| | Working | Working | Working | Working |
|---|---|---|---|---|
| | Ex. 6 | Ex. 7 | Ex. 8 | Ex. 9 |
| Hydroxypropyl methyl | 5.0 | 5.0 | 5.0 | 5.0 |
| cellulose phthalate | | | | |
| Diisopropyl adipate | — | 5.0 | 12 | — |
| Diisopropyl sebacate | — | — | — | 5.0 |
| Povidone-iodine | 25 | 25 | 25 | 25 |
| (40% ethanol solution) | | | | |
| Ethanol | 39 | 39.5 | 40.2 | 39.5 |
| Water | 30.4 | 24.9 | 17.2 | 24.9 |
| Potassium hydroxide | 0.6 | 0.6 | 0.6 | 0.6 |
| aqueous solution (4M) | | | | |
| Total | 100 | 100 | 100 | 100 |

Drape adhesion tests were performed as follows for the compositions of Working Examples 6 to 9 and Reference Examples 1 and 2. Each composition was impregnated in a foam and applied to pig skin cut and divided in advance. After application, the composition was left to dry for 20 minutes. An Ioban (registered trademark) 2 Antimicrobial Incise Drape (available from the 3M Company) was cut into a strip shape with a width of 0.5 inches (1.27 cm), and after this was adhered to the dried composition, the composition was crimped with a 4.5 pound (2.1 kg), 2 inch (5.1 cm) roller in order to realize a uniform adhesive pressure. Each strip was left to stand for 30 minutes in order to generate an appropriate amount of adhesion. Gauze immersed in saline was then adhered to the upper part of the strip, and this was left to stand for 20 minutes to produce a liquid loading environment. Next, each strip was peeled at a peeling angle of 90° and a speed of 12 inches/min (30.5 cm/min) using a Zwick Roell 7005 tensile strength measurement device. The average value of the strength (N) required to peel each sample over a length of at least 3 inches (7.6 cm) was recorded. At least four strips were evaluated for each composition, and the average values of the measured strengths are shown in Table 7.

TABLE 7

| | Drape adhesive strength |
|---|---|
| | (Newtons) |
| Working Example 6 | 0.0822 |
| Working Example 7 | 0.2581 |
| Working Example 8 | 0.3953 |
| Working Example 9 | 0.2478 |
| Reference example 1 | 0.2396 |
| Reference Example 2 | 0.5438 |

As shown in Table 5, the drape adhesive strength of Working Example 2 was superior to the drape adhesive strength of Comparative Examples 2 and 4 and Reference Example 1.

### Bactericidal Action Test

The bactericidal action was confirmed using the composition of Working Example 3, the composition of Reference Example 1, and 3M One-step Patient Prep (7.5% povidone-iodine aqueous solution, available from the 3M Company; Reference Example 3). The abdominal skin of a euthanized pig was divided into a lattice of approximately 2.5 inches×2.5 inches, and each composition was applied. For the application site of each composition, at least three sections were selected at random for each composition, and the application sites were equally and continuously assigned from the upper part and lower part of the abdominal skin. The application method used for the bactericidal composition of Reference Example 1 was a conventional application method for treatment room environments in Japan in which gauze immersed in a bulk solution is applied. That is, the composition was applied with gauze so as to draw a circle from roughly the center of the skin segment toward the outside, and this was repeated three times. In addition, in the application method of the composition of Reference Example 3, the composition was applied to the skin of a pig by using a unique one-step applicator system. The composition of Working Example 3 was applied to the skin surface using a one-step applicator system having a barrel for storing a solution and a foam sponge adhered to the upper part of the barrel filled with the solution, and this functioned as a holder for the bactericidal composition during prepping. That is, both the compositions of Reference Example 3 and Working Example 3 were applied by rubbing the compositions over the skin for 30 seconds using a foam sponge so that the same locations may be overlapped based on the design specifications and user manual of the applicator.

After each composition was applied to the application site using gauze or using a foam sponge, the composition was left to stand for 10 minutes until completely dry. The surviving bacteria were then sampled using a “cup scrub method” (ASTM E1874), and the colonies of surviving bacteria were counted. That is, an aseptic scrub cup was placed on the skin to which the composition was applied, and after 2.5 mL of a sampling solution was added, the site was scrubbed for 1 minute with a rubber policeman (plastic scrub stick). After one minute, the sampling solution was transferred to a 15 mL tube. Each step was repeated in a similar manner so that the amount of the sampling solution was ultimately 5 mL. The number of surviving bacteria of the negative control (baseline) was sampled and counted with the same method at three untreated skin sites. After each sample was diluted in a stepwise manner (10 times, 100 times, and 1000 times), the sample was cultured on a plate. Next, the cultured sample was incubated for 3 days at 37° C., and the colonies were counted. The common logarithm values (Log CFU/mL) of the resulting numbers of surviving bacteria are shown in Table 8.

TABLE 8

| | | CFU Log Recovery | |
|---|---|---|---|
| | | of Pig Skin Flora | Std. |
| Example | Replicates | after Treatment | Dev. |
| Negative Control (Baseline) | 3 | 4.43 | 0.48 |
| Reference example 1 | 6 | 3.22 | 0.74 |
| Reference Example 3 | 3 | 3.23 | 0.45 |
| Working Example 3 | 6 | 2.64 | 0.62 |

The composition of Working Example 3 demonstrated a bactericidal action superior to that of Reference Examples 1 and 3.

### Compatibility Test

Hydroxypropyl methyl cellulose phthalate, a plasticizer (diisopropyl adipate, tri(capryl/caprylic acid)glycerin, or isopropyl myristate), povidone-iodine, and ethanol were mixed in accordance with the content of Table 9 or Table 10. Next, the appearance of the composition when water was added and stirred was observed.

TABLE 9

| | Working | Comp. | Comp. | Comp. | Comp. |
|---|---|---|---|---|---|
| | Ex. 10 | Ex. 6 | Ex. 7 | Ex. 8 | Ex. 9 |
| Hydroxypropyl | 5.0 | 5.0 | 5.0 | 5.0 | 5.0 |
| methyl cellulose | | | | | |
| phthalate | | | | | |
| Diisopropyl adipate | 12 | — | — | — | — |
| Tri(capryl/caprylic | — | 2.0 | 4.0 | 6.0 | 8.0 |
| acid)glycerin | | | | | |
| Povidone-iodine | 25 | 25 | 25 | 25 | 25 |
| (40% ethanol solution) | | | | | |
| Ethanol | 40.2 | 39.2 | 39.4 | 39.6 | 39.8 |
| Water | 17.8 | 28.8 | 26.6 | 24.4 | 22.2 |
| Total | 100 | 100 | 100 | 100 | 100 |

TABLE 10

| | Comp. | Comp. | Comp. | Comp. |
|---|---|---|---|---|
| | Ex. 10 | Ex. 11 | Ex. 12 | Ex. 13 |
| Hydroxypropyl methyl | 5.0 | 5.0 | 5.0 | 5.0 |
| cellulose phthalate | | | | |
| Isopropyl myristate | 2.0 | 4.0 | 6.0 | 8.0 |
| Povidone-iodine | 25 | 25 | 25 | 25 |
| (40% ethanol solution) | | | | |
| Ethanol | 39.2 | 39.4 | 39.6 | 39.8 |
| Water | 28.8 | 26.6 | 24.4 | 22.2 |
| Total | 100 | 100 | 100 | 100 |

TABLE 11

| | Solubility |
|---|---|
| Working Example 10 | Dissolved |
| Comparative Example 6 | Insoluble with white turbidity |
| Comparative Example 7 | Insoluble with white turbidity |
| Comparative Example 8 | Insoluble with white turbidity |
| Comparative Example 9 | Insoluble with white turbidity |
| Comparative Example 10 | Insoluble with white turbidity |
| Comparative Example 11 | Insoluble with white turbidity |
| Comparative Example 12 | Insoluble with white turbidity |
| Comparative Example 13 | Insoluble with white turbidity |

The results are shown in Table 11. In Working Example 10 containing diisopropyl adipate, a colorless, clear composition was obtained, but in Comparative Examples 6 to 9 containing tri(capryl/caprylic acid)glycerin and Comparative Examples 10 to 13 containing isopropyl myristate, the ingredients did not dissolve, and the compositions became white and turbid.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A bactericidal composition comprising a carboxy group-containing cellulose ester or carboxy group-containing cellulose ether, a bactericide, alcohol, and water.

2. The bacterial composition according to claim 1 wherein the composition contains a carboxy group-containing cellulose ester which is a dicarboxylic acid monoester of hydroxyalkyl alkyl cellulose.

3. The bactericidal composition according to claim 2, wherein the composition contains a carboxy group-containing cellulose ester taken from the group consisting of hydroxypropyl methyl cellulose phthalate and hydroxypropyl cellulose acetate succinate.

4. The bactericidal composition according to claim 3, wherein the carboxy group-containing cellulose ester is hydroxypropydl methyl cellulose phthalate.

5. The bacterial composition according to claim 1, wherein the composition contains a carboxy group-containing ether which is a hydroxyalkyl alkyl cellulose.

6. The bacterial composition according to claim 5, wherein carboxy group-containing ether is carboxymethyl cellulose.

7. The bactericidal composition according to claim 1, further comprising a dicarboxylic acid ester.

8. The bacterial composition according to claim 1, wherein the bactericide is taken from the group consisting of iodine, triiodide salts, and iodophors.

9. The bactericidal composition according to claim 8, wherein the bactericide is an iodophor.

10. The bactericidal povidone-iodine composition according to claim 9, wherein the bactericide is povidone-iodine.

11. The bacterial composition according to claim 1, wherein the ester or ether further contains an aromatic ring.

12. The bactericidal composition according to claim 2, further comprising a dicarboxylic acid ester.

13. The bactericidal composition according to claim 3, further comprising a dicarboxylic acid ester.
